(19) United States
(12) Patent Application Publication
(10) Pub. No.: US 2024/0269477 A1
(43) Pub. Date: Aug. 15, 2024

(54) PORTABLE TEETH WHITENING APPARATUS

(71) Applicant: Marc A. Pickren, Lakeway, TX (US)

(72) Inventor: Marc A. Pickren, Lakeway, TX (US)

(21) Appl. No.: 18/108,323

(22) Filed: Feb. 10, 2023

(51) Int. Cl. A61N 5/06

(52) U.S. Cl. CPC A61N 5/0603; A61N 2005/0606; A61N 2005/065

(57) ABSTRACT

A portable teeth whitening device comprising a housing having a light source and a power source therein. The light source is designed to emit light through a removable mouthpiece to the teeth of a user during a whitening session. A microcontroller controls the output of the light source according to one or more lighting schemes to optimize whitening treatments.

### FIELD OF THE INVENTION

This invention relates to teeth whitening. More specifically, this invention is related to a portable teeth whitening apparatus.

### BACKGROUND

Teeth can become yellow due to aging or consumption of food, drink, or tobacco. White teeth are often perceived as being more attractive and having whiter teeth can improve many areas of an individual's life, including self-confidence, career opportunities, and romantic relationships.

To this end, many solutions have been developed to whiten teeth. These solutions have varying effectiveness; however, most solutions involve the application of hydrogen peroxide or carbamide peroxide to the surface of teeth and waiting a length of time for the peroxides to whiten the teeth. Treatments fall into two main categories: treatments that can be performed by a user at home and in-office treatments that must be administered by a dental professional at their office.

Home whitening with peroxides alone can take up to several hours per day over the course of weeks, which is inconvenient for the user. To shorten the time required to whiten teeth, electromagnetic waves can be applied to the teeth in the form of visible or ultraviolet light. Commercial light systems have been developed which are administered in-office by a dental professional which can whiten teeth in under an hour. These commercial light systems are large and expensive and are therefore only suited for dental professionals. Making appointments and visiting dental professionals can be costly and a hassle for consumers.

It would therefore be advantageous to have a teeth whitening apparatus which avoids these and other drawbacks of existing devices.

### SUMMARY OF THE INVENTION

A portable teeth whitening apparatus according to embodiments may comprise a main unit comprising a housing having an internal cavity therein and a light source within the housing for emitting light to be applied to teeth of a user. The apparatus may also comprise a detachable mouthpiece that is removably connectable to the main unit, the mouthpiece designed to fit in the mouth of a user and allow at least a portion of light emitted from the light source to pass through the mouthpiece to the teeth of the user.

The mouthpiece may be made of a transparent or translucent material such as silicone rubber. A reflective ring may be included in the mouthpiece to contain light and direct it to the teeth.

The mouthpiece may comprise a light conducting portion and a light blocking portion, the light conducting portion comprising a first material that is translucent and the light blocking portion comprising a second material that is opaque. The first and second material may be silicone rubber and the second material may be white.

The mouthpiece may also comprise a tubular section for interfacing with the main unit and a U-shaped section to be placed in the user's mouth. The U-shaped section may comprise an outside flange and the light blocking portion may extend along an outer surface of the tubular section and along an outer surface of the outside flange.

The mouthpiece may further comprise a locking flange designed to secure the mouthpiece to the main unit.

A portable teeth whitening apparatus according to other embodiments may comprise a housing having an internal cavity therein, a light source within the housing for emitting light to be applied to teeth of a user, and a mouthpiece connected to the housing. The mouthpiece may be designed to fit in the mouth of a user and allow at least a portion of light emitted from the light source to pass through the mouthpiece to the teeth of the user. The light source may be designed to emit light according to a lighting scheme, the lighting scheme defining the intensity of the light source during a treatment session. The lighting scheme may have a variable intensity during the treatment session.

The lighting scheme may comprise a repeating waveform such as a square, sine, sawtooth, or triangle wave. The waveform may have a wavelength of one to three seconds. More specifically, the waveform may be a square wave with a wavelength of two seconds, the light being at a first intensity for one second and at zero intensity for one second.

The apparatus may comprise a plurality of lighting schemes and the user may select a lighting scheme from the plurality of lighting schemes.

A safety sensor may be included to reduce or halt light output of the light source when the portable teeth whitening apparatus is not in proximity to the user.

### BRIEF DESCRIPTION OF THE DRAWINGS

FIG. 1A depicts a view of a portable teeth whitening apparatus with the mouthpiece separated according to one embodiment.

FIG. 1B depicts a view of the portable teeth whitening apparatus of FIG. 1A with the mouthpiece installed.

FIG. 1C depicts an exploded view of the portable teeth whitening apparatus of FIG. 1B.

FIG. 1D depicts a section view of the portable teeth whitening apparatus of FIG. 1B.

FIG. 2A depicts a front view of a printed circuit board of a portable teeth whitening apparatus according to one embodiment.

FIG. 2B depicts a rear view of the printed circuit board of FIG. 2A.

FIG. 3 depicts a block diagram of the electrical components of a portable teeth whitening apparatus according to one embodiment.

FIG. 4A depicts a perspective view of a mouthpiece according to one embodiment.

FIG. 4B depicts a section view of the mouthpiece of FIG. 4A.

FIG. 4C depicts another section view of the mouthpiece of FIG. 4A.

FIG. 4D depicts an exploded view of the mouthpiece of FIG. 4A.

FIG. 5 depicts a sectional view of a portion of a teeth whitening apparatus according to an embodiment.

FIG. 6 depicts a sectional view of a portion of a teeth whitening apparatus according to an embodiment.

FIG. 7 depicts a sectional view of a portion of a teeth whitening apparatus according to an embodiment.

FIG. 8 depicts a mouthpiece according to an embodiment.

FIG. 9 depicts a mouthpiece according to an embodiment.

### DETAILED DESCRIPTION

Home teeth whitening treatments can take many hours per day for several weeks. In-office teeth whitening systems can whiten teeth faster, however, visiting a dental professional is inconvenient and costly. To overcome these and other drawbacks of existing devices, a portable teeth whitening apparatus is disclosed herein.

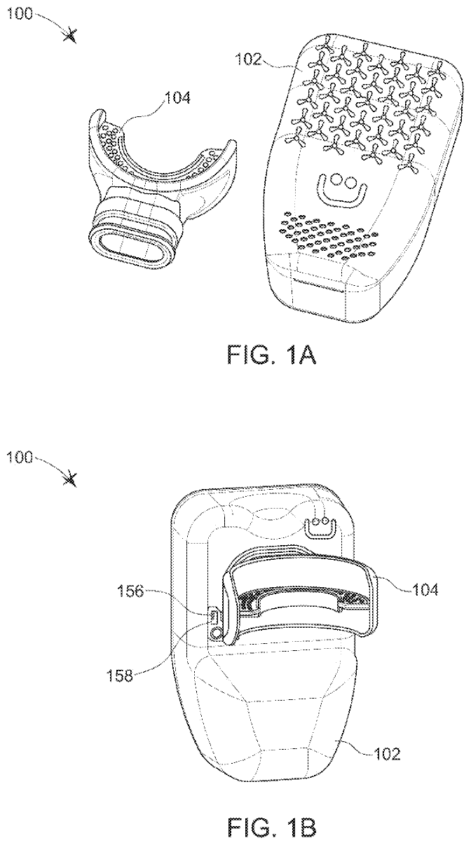

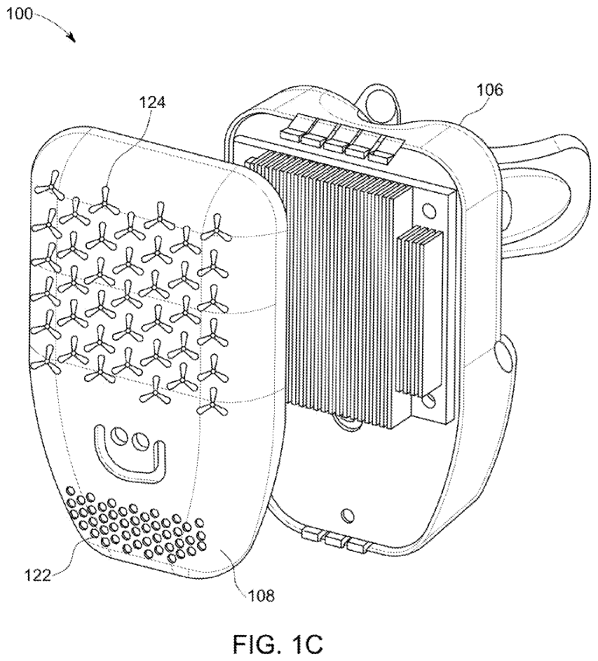

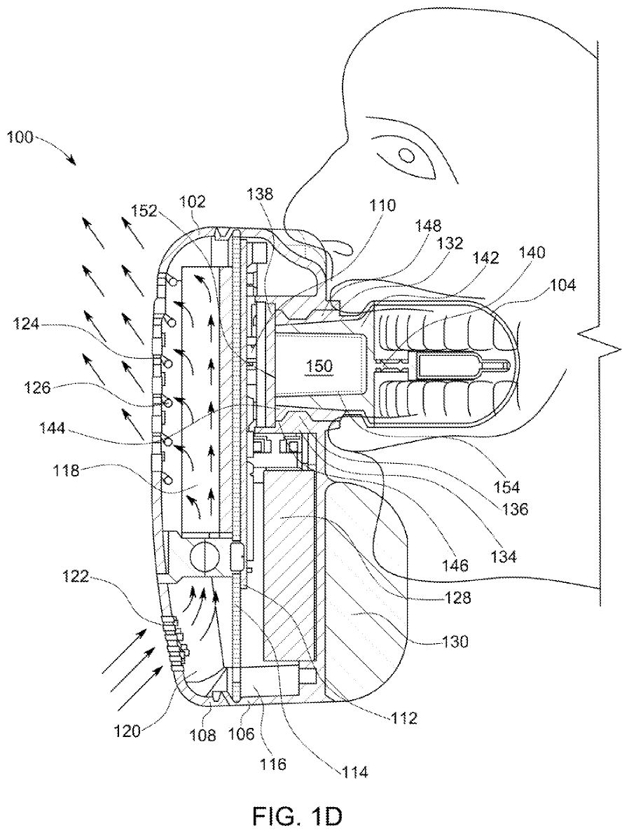

FIGS. 1A-1D show an embodiment of a portable teeth whitening apparatus 100. Portable teeth whitening apparatus 100 is comprised of a main unit 102 and a removable mouthpiece 104. Main unit 102 contains a light source, a power source, and other electronic components. Main unit 102 is comprised of a two-piece housing that contains and protects the components therein. The two-piece housing is comprised of an inward-facing housing section 106 that faces the user when in use and an outward-facing housing section 108 that faces away from the user when in use. Housing sections 106 and 108 are formed of a polymer such as ABS, polycarbonate, or nylon, metal such as aluminum, steel, stainless steel, or copper, or any other suitable material and may be injection molded, machined, or formed by additive manufacturing. Outward-facing housing section 108 may be attached to the rest of the unit via snap joint or other removable means to allow a user to customize the appearance of the device by replacing outward-facing housing section 108 with one of a different design.

A light source 110 is contained within the housing and is configured to apply light to the teeth of a user through mouthpiece 104 to facilitate the whitening process. The light source may be any known source of light, including one or more Light Emitting Diodes (LEDs). The light source may produce light having a wavelength of between 400 and 505 nanometers (nm), which is in the blue/violet range of the visible spectrum and has been shown to be effective in whitening teeth coated in whitening gel. Ultraviolet wavelengths (i.e. below about 400 nm) may also be effectively used in whitening teeth. The light source is not limited to just these wavelengths, however. Any other wavelength or combination of wavelengths may be used. For example, a light source may comprise an LED having a wavelength of 445 nm (blue/violet) and an LED having a wavelength of 380 nm (ultraviolet).

Light source 110 is connected to a Printed Circuit Board (PCB) 112, which will be described in greater detail later. Printed circuit board 112 is mounted to a metallic plate 114 which may be made of metal including aluminum or copper, or any other suitable material. A thermally conductive paste, adhesive, or other material may be used between PCB 112 and metallic plate 114 to facilitate heat transfer from the light source and other components on the PCB to prevent thermal damage to the light source, PCB components, or the rest of the apparatus. Metallic plate 114 is a flat plate shaped to fit within a corresponding opening in housing section 106 to create a sealed cavity 116 within housing section 106. An adhesive or O-ring may be used at the intersection of metallic plate 114 and housing section 106 to improve the resistance to water and dust intrusion into sealed cavity 116. Thus, sealed cavity 116 may be water resistant or waterproof to protect the components from damage. PCB 112 may comprise a conformal coating in order to provide further resistance to moisture.

Attached to metallic plate 114 opposite the side of PCB 112 is a dissipator 118 which is in thermal communication with metallic plate 114. Dissipator 118 may be attached using thermally conductive tape or adhesive. Dissipator 118 may also be held by mechanical fasteners, adhesives, or other means and may have a thermally conductive paste. To provide airflow to dissipator 118, a plurality of openings are formed in outward-facing housing section 108. Because of these openings, the space formed within housing section 108 (in contrast to the sealed cavity 116) is an unsealed cavity 120. Lower openings 122 may have a first shape and may act primarily as an intake for air to enter the unsealed cavity 120. Upper openings 124 have a second shape and may act primarily as an exit for air within the unsealed cavity 120. Convection powered by the heat from metallic plate 114 and dissipator 118 causes air to be drawn into lower openings 122 where it absorbs heat and is exhausted through upper openings 124 as shown by the arrows. An airflow guide 126 comprising one or more louvers directs hot air away from a user's face to prevent discomfort. Alternatively, in another embodiments, an airflow guide may be used in conjunction with appropriately placed openings such as along the top edge to use the air inhaled and exhaled from the nose as forced convection to aid in cooling.

Moving back to sealed cavity 116, a power source 128 in the form of a rechargeable battery is provided. Power source 128 is connected to the PCB 112 to provide power for the light source and for the operation of the device.

A chin pad 130 is attached to housing section 106 below the location of an attached mouthpiece. Chin pad 130 is made of a rubber, foam, or other soft material to provide comfortable support against the chin of a user when the device is being used. Chin pad 130 may be overmolded onto housing section 106, adhered with an adhesive, or mechanically fastened, for example, by having an enlarged section protruding through a smaller opening.

Above chin pad 130 is mouthpiece socket 132 in the form of a cavity in the housing section 106. Mouthpiece socket 132 is configured to receive and removably retain mouthpiece 104. Mouthpiece socket 132 comprises a tube-like thin-walled projection 134 extending from the outer surface of housing section 106 and designed to encircle mouthpiece 104. Projection 134 comprises an asymmetric ledge 136 which is sloped more greatly on one side than the other side. Mouthpiece 104 will therefore slide easily into place in mouthpiece socket 132 but resist removal during use.

A translucent barrier 138 defines the bottom of the mouthpiece socket 132. Translucent barrier 138 is attached to projection 134 and may be overmolded or integrally formed with housing section 106 or may be sealed with an O-ring, adhesive, or sealant to prevent unwanted material from entering sealed cavity 116. Light source 110 is within sealed cavity 116 and aligned with mouthpiece 104 and translucent barrier 138 in order to apply light to teeth for whitening. Translucent barrier 138 may include lenses, collimators, or otherwise be shaped so as to direct light to desired areas. Translucent barrier 138 may be transparent or semi-transparent and may be water-clear or optically clear.

Due to the high intensity of light source 110, housing section 106 may be made of an opaque material to contain stray light. Additionally, projection 134 extends beyond translucent barrier 138 to create a dual-barrier to prevent light from leaking through housing section 106. That is, light is contained first within projection 134 and any light leaking through is contained within sealed cavity 116 by housing section 106 and metallic plate 114. Light therefore exits through translucent barrier 138 only.

Mouthpiece 104 is comprised of a thin-walled body made of a non-toxic, flexible translucent or transparent material such as medical grade rubber or silicone. Mouthpiece 104 is comprised of a U-shaped section designed to be placed in a user's mouth and a tubular section designed to interface with mouthpiece socket 132. The U-shaped section is comprised of a bite section 140 having a planar shape that is designed to fit between upper and lower teeth of a user. An outside flange 142 extends perpendicular to the bite section and extends along the front side of the user's teeth. The outside flange 142 is designed to be placed between the user's teeth and lips/cheek. The tubular section is a hollow portion comprised of an outer thin-walled ring having a shape designed to fit snugly within the mouthpiece socket 132. A locking flange 144 extends perpendicularly from the end of the tubular section and interfaces with locking groove 146 which is an annular groove defined by mouthpiece socket 132, asymmetric ledge 136, and translucent barrier 138. Mouthpiece 104 also has a secondary flange 148 to aid in stability and help the tubular section maintain shape. Inner cavity 150 of the tubular section of the mouthpiece is a hollow portion having an opening 152 at one end and is designed to let light pass through to the teeth. A reflective ring 154 is comprised of a thin tubular section attached to the side walls of the interior cavity 150 in order to help contain light from escaping the mouthpiece, which improves efficacy and the user experience. Reflective ring 154 may be a shiny or mirrored piece or it may comprise a coating applied to the mouthpiece. Both ends of the reflective ring 154 are open to allow light to enter and exit the reflective ring. Alternatively, the reflective ring may be incorporated into the main unit 102 instead of the mouthpiece.

The mouthpiece is designed to help distribute light evenly to the teeth with minimal leakage of light outside of the mouth or to other parts of the mouth. The mouthpiece is shaped to fit comfortably in the mouth of a user for the duration of the treatment session. Because the mouthpiece is removable and replaceable, the portable teeth whitening apparatus may be used by several users, with each user having their own mouthpiece.

A safety sensor 156 in the form of a proximity sensor (PS), ambient light sensor (ALS), or high power IRED is used to detect that the device has been placed in the mouth of a user. The sensor may incorporate photodiodes, amplifiers, and analog to digital converting circuits into a single chip by CMOS process. The safety sensor may take other forms such as a camera, infrared emitter/receiver, ultrasonic, LIDAR, temperature sensor, capacitive, or physical switch. The safety sensor may be mounted within the sealed cavity 116 proximal to a safety sensor opening 158 in the housing section 106. A seal in the form of an adhesive, sealant, gasket, or O-ring may be used to seal safety sensor opening 158.

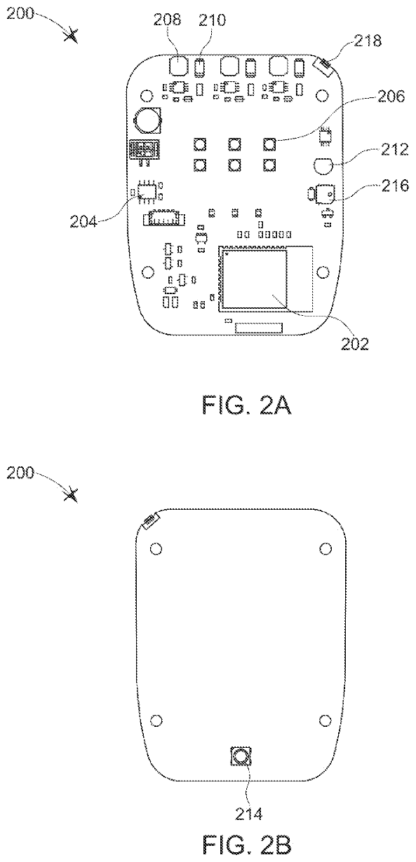

FIG. 2 shows a Printed Circuit Board (PCB) 200 according to one embodiment. PCB 200 may be made of glass reinforced epoxy laminate, aluminum, copper, or other metal, or any other suitable material. Mounted on PCB 200 is a programmable microcontroller 202 for operating the device. The microcontroller 202 is powered by a power source in the form of an AC or DC external power source or a battery. A voltage regulator may be used to raise or lower the voltage from the power source to a desired voltage for powering the microcontroller, light source, and other devices. Light source 206 in the form of six LEDs is mounted in the center of PCB 200, though any number of LEDs is possible. The LEDs are driven by LED drivers 208 which receive power from power supply 204 and are controlled by microcontroller 202. One or more current sensors 210 may be connected to the LED circuit for monitoring and limiting the current supplied to the LEDs to prevent damage to the LEDs or other components of the device. Additionally, a temperature sensor 212 is mounted proximal to the LEDs and is connected to the microcontroller to provide temperature information to the microcontroller. The microcontroller may reduce or shut off power to the LEDs above a specified temperature limit. The microcontroller may generate a visual or audible warning or alarm to alert the user if an overheating condition is imminent or has occurred. Visual indications or alarms may be indicated by status light 214, which may be a multi color or RGB LED connected to and controlled by microcontroller 202. The status light 214 may also be used to illuminate the product name or logo which may be incorporated into the outward-facing housing section 108 in the form of an opening, thinner section, translucent, or transparent portion.

Audible indications or alarms may be indicated by buzzer 216 connected to and controlled by microcontroller 202. The buzzer may indicate the device has been turned on or off, selection of a treatment session as well as the start, end or pause of a treatment session, low battery, fully charged battery, or alarm for undesired condition such as overheating.

The portable teeth whitening apparatus may control the light source during a treatment session according to a lighting scheme. A treatment session is defined as an event beginning with the initial activation of the light source and continuing until the maximum duration of the lighting scheme is reached or alternatively before the maximum duration is reached by the user ending the session or if the apparatus shuts down for any other reason such as exhausted battery or overheating. A lighting scheme defines the maximum duration of the treatment session (i.e., 20 minutes), as well as the intensity of the light at each point in time. The lighting scheme may have a constant intensity or variable (non-constant) intensity throughout the session. In other words, the lighting scheme may comprise a constant brightness for the entire duration of the session, or the lighting scheme may comprise two or more intensity levels during the session. For example, the light intensity may increase or decrease over the length of the session. The change in intensity may be linear or follow a curved path such as a parabola or exponential curve. The light intensity may have a repeating pattern at a fixed or variable frequency. For example, the light intensity may have the waveform of a sine, square, triangle, sawtooth, ½ sine, exponential decay, or any other waveform. The waveform may have one or more wavelengths throughout the lighting scheme. Any wavelength may be used. The wavelength may preferably be between 0.1 and 30 seconds and even more preferably between 1 and 3 seconds. The treatment session may include a portion of steady illumination and a portion of patterned illumination. Lighting schemes may be designed based on user preferences (e.g., time, sensitivity, etc.) or based on the whitening agent used (e.g., hydrogen peroxide, carbamide peroxide; % concentration) or the user's teeth color.

Lighting schemes may be defined which have an advantageous effect. For example, a lighting scheme may be created for a normal treatment session. The scheme may run for a period of time such as 20 minutes and may limit the light output of the light source to reduce discomfort. Another lighting scheme may be created which maximizes the whitening for a shorter duration. Such a scheme may run for 5 or 10 minutes and maximize light output to accomplish the most whitening in the short session. Yet another lighting scheme may use a greatly reduced light output for users who have greater than normal sensitivity and experience greater discomfort during treatment. Different lighting schemes may also be provided for initial whitening and regular maintenance. Lighting schemes may be optimized by AI (Artificial Intelligence) algorithm that receives information such as current whiteness level from a sensor or from a user input.

A whiteness sensor may be built into the whitening apparatus and the device may take whiteness readings before, during, or after a treatment session to monitor and report teeth whiteness and also to control whitening sessions. For example, the whiteness sensor may be attached to the circuit board and may receive an image or whiteness level from the teeth. Using the whiteness sensor, a value may be assigned to the whiteness of the teeth at specified intervals. For example, the whiteness can be measured at the beginning of the treatment session and periodically during the treatment session as well as at the end of the treatment session. The device may be programmed to end a treatment session when a certain change in whiteness has been achieved. The device may store and transmit the whiteness data to another device. The device may use the teeth whiteness data to show a chart or graph of the user's teeth whiteness over time. Whiteness may also be received based on an image taken by a phone, tablet, digital camera, or other imaging device. The image may be analyzed by an app on the device and may send whiteness information to the apparatus. A user may also make a manual measurement of whiteness and input the whiteness value obtained into an app or directly into the apparatus.

Lighting schemes provide several benefits. They allow the user to customize the treatment based on their preferences. The effectiveness of the treatment session can be maximized while limiting the power needed to run the device. By limiting the power, battery life can be extended, or a smaller battery may be used which decreases cost and increases user comfort. Additionally, the light source produces less waste heat which reduces the need for cooling and increases user comfort. Further, the risk of the light itself causing discomfort or injury is reduced. Increasing the temperature in the mouth as little as 11 degrees Fahrenheit can cause permanent nerve damage.

In experimental testing, it was found that a pulsing lighting scheme of 100% intensity with 50% duty cycle square wave having a two second wavelength resulted in better whitening performance than a steady scheme of 100% intensity for the same duration. Additionally, experimental testing showed using a light source with whitening gel produced 1900% greater whitening than with gel alone. Thus, a portable teeth whitening apparatus as disclosed herein can provide better performance than traditional home whitening without needing to visit a dental office for treatment.

An optional fan (not shown) may be connected to PCB 202 via a fan output connector to assist in cooling the device. The fan may be within the unsealed cavity and be configured to move air across the metallic plate or dissipator or to move air into or out of the unsealed cavity. A current sensor for the fan may be included to allow the microcontroller to monitor and limit the current sent to the fan to prevent damage or other unwanted conditions.

At least one button 218 for user control is connected to the microcontroller 202. The button 218 may allow the user to turn the device on or off, select modes or other settings, and start, pause, or end a treatment session. The user may pause the treatment session and resume the session within a set amount of time. In such a case, the treatment may resume from the point in the lighting scheme where the treatment was paused. If too much time elapses before the session is resumed, the apparatus may end the session and turn off.

A safety sensor (not shown in FIG. 2) may be connected to the microcontroller to detect the presence of a user. The safety sensor is connected to the microcontroller 202 to send either a positive or negative indication of a user's presence and may reduce or halt light output and pause the session if a negative indication is received in order to reduce the likelihood of shining light into a person's eyes.

The microcontroller may also be connected to a wireless communication device that allows the apparatus to send and receive data or commands via a wireless connection. A device such as a smartphone, tablet, or computer may be used to select settings, start, pause, or end a session, as well as receive whiteness data from the whiteness sensor, session data, and device status. Session data may include treatment time elapsed or remaining, most recent whiteness reading, etc. Device status may include battery state of charge, low battery, overheating conditions, or other errors or malfunctions.

To dissipate heat and reduce the likelihood of thermal shutdown or damage, PCB 200 has a plurality of thermal vias (not shown), particularly near the light source 206 and drivers 208. The thermal vias assist in transferring heat to the opposite side of the board, which is in thermal communication with the metallic plate and dissipator. In other embodiments, PCB 200 may be made of a thermally conductive material such as aluminum, copper, or other metal. Due to these thermal management features, the portable teeth whitening apparatus may be capable of meeting or exceeding the light output levels of professional devices in a compact portable form factor.

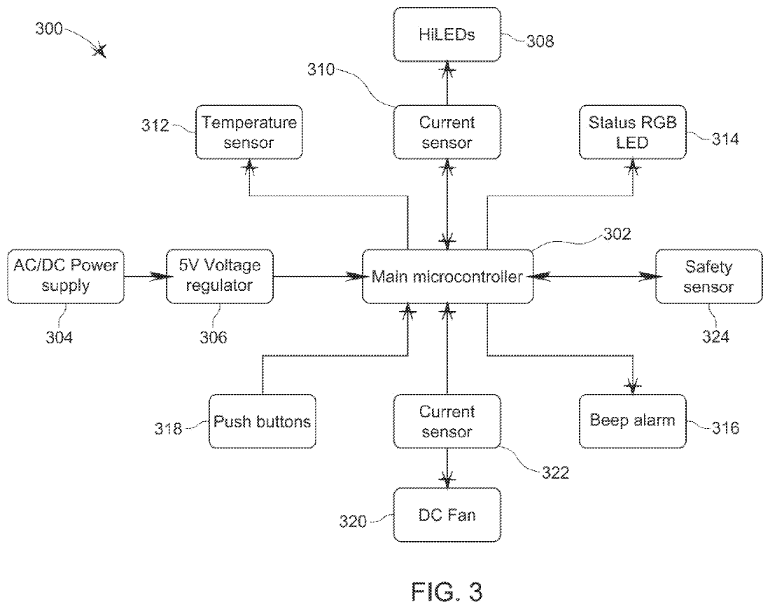

FIG. 3 depicts a block diagram of the electrical components of a portable teeth whitening apparatus according to one embodiment. Microcontroller 302 is powered by a power source 304 in the form of an AC or DC external power source or a battery. A voltage regulator 306 may be used to raise or lower the voltage from the power source 304 to a desired voltage for powering the microcontroller, light source, and other devices. Light source 308 is driven by LED drivers which receive power from power supply 304 and are controlled by microcontroller 302. One or more current sensors 310 may be connected to the LED circuit for monitoring and limiting the current supplied to the LEDs to prevent damage to the LEDs or other components of the device. Additionally, a temperature sensor 312 is mounted proximal to the LEDs and is connected to the microcontroller 302 to provide temperature information to the microcontroller 302. The microcontroller 302 may reduce or shut off power to the LEDs above a specified temperature limit. The microcontroller may generate a visual or audible warning or alarm to alert the user if an overheating condition is imminent or has occurred. Visual indications or alarms may be indicated by status light 314, which may be a multi color or RGB LED connected to and controlled by microcontroller 302. Audible indications or alarms may be indicated by buzzer 316 connected to and controlled by microcontroller 302. At least one button 318 for user control is connected to the microcontroller 302. The button 318 may allow the user to turn the device on or off, select modes or other settings, and start, pause, or end a treatment session. Fan 320 may be connected to microcontroller 302 to assist in cooling the device. A current sensor 322 may sense the current in order to limit the current sent to the fan to prevent damage to the unit. A safety sensor 324 may be connected to the microcontroller 302 to detect the presence of a user's face to prevent the light source from activating in undesired situations.

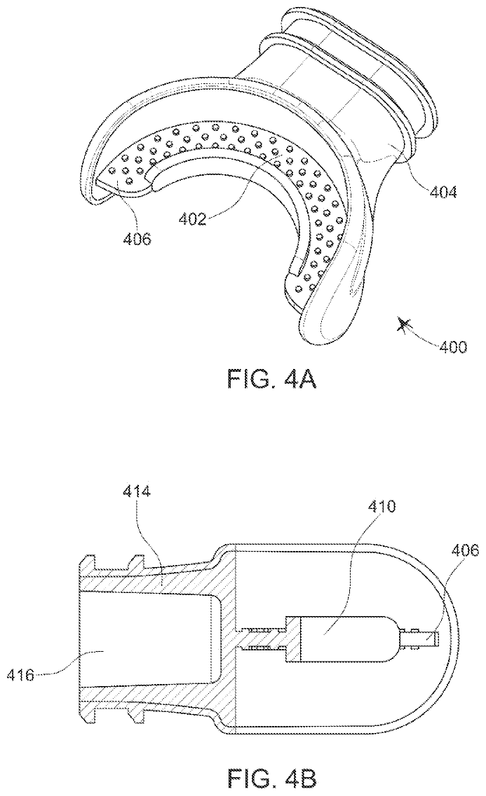

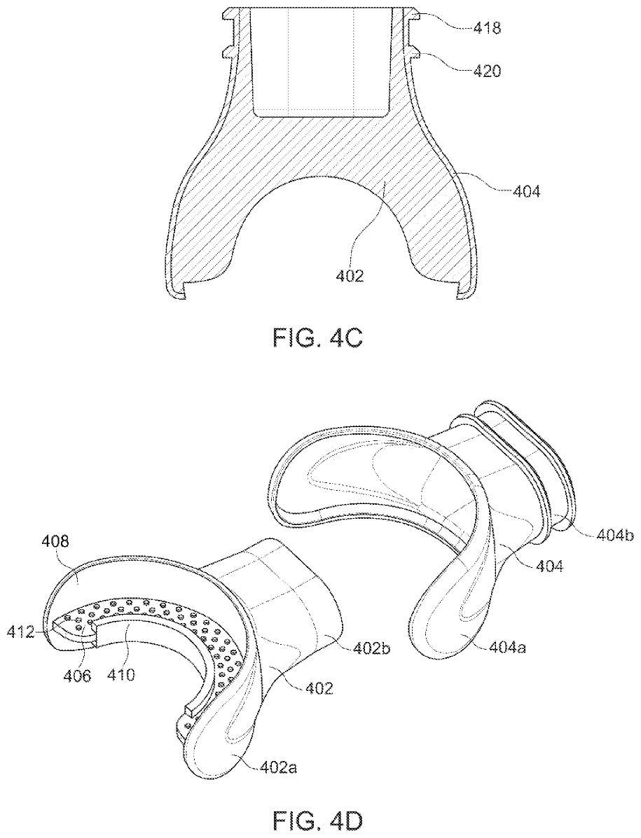

FIGS. 4A-D depict a mouthpiece for a teeth whitening apparatus according to one embodiment. FIG. 4A depicts a perspective view of a mouthpiece according to one embodiment. FIG. 4B depicts a section view of the mouthpiece of FIG. 4A. FIG. 4C depicts another section view of the mouthpiece of FIG. 4A. FIG. 4D depicts an exploded view of the mouthpiece of FIG. 4A.

Mouthpiece 400 is comprised of a transparent or translucent (semi-transparent) light conducting portion 402 and an opaque or nearly opaque light blocking portion 404. Light conducting portion 402 is designed to transmit and disperse light from the light source of the main unit to the teeth of a user. Light blocking portion 404 is designed to prevent light from escaping the mouthpiece in undesired directions. Both light conduction portion 402 and light blocking portion 404 are comprised of a U-shaped section (402a and 404a, respectively) designed to be placed in a user's mouth and a tubular section (402b and 404b, respectively) designed to interface with a mouthpiece socket of a main unit.

The U-shaped section 402a of the light conduction portion 402 is comprised of a bite section 406 having a planar crescent or partial ring shape that is designed to fit between upper and lower teeth of a user. An outside flange 408 extends perpendicular to and along the outer circumference the bite section 406 and is designed to extend along the front side of the user's teeth. In use, outside flange 408 is placed between the user's teeth and lips/cheek. When in place, the outside flange 408 is naturally held against the user's teeth and provides support for the teeth whitening apparatus. An inside flange 410 extends perpendicular to and along the inner circumference of the bite section 406. Inside flange 410 may aid in placing the mouthpiece in the proper position and retain it there. Inside flange 410 may also assist in supporting the teeth whitening apparatus and distributing light to the teeth. Bite section 406 may have a texture 412 where upper and lower teeth contact in order to increase comfort. Texture 412 may take various forms such as bumps, ribs, etc.

Tubular section 402b of the light conduction portion 402 is a hollow section attached to the U-shaped section 402a and is comprised of an outer thin-walled tube 414 having an inner cavity 416 having an opening at one end. Tube 414 is designed to let light pass through the tubular section 402 of the mouthpiece to the teeth with minimal loss. Because the tubular section 402b is comprised of a transparent material, light entering the tube 414 may pass directly through the material to the teeth or may be reflected through total internal reflection to the teeth. The shape of the surfaces of the light conducting portion 402 may be designed to direct light to desired areas.

Light blocking portion 404 covers the outer surface of tube 414 and the outer surface of outside flange 408. Tubular section 404b has a shape designed to fit snugly within a mouthpiece socket. A locking flange 418 extends perpendicularly from the end of the tubular section 404b and interfaces with a locking groove of a teeth whitening apparatus main unit. Light blocking portion 404 also has a secondary flange 420. Both locking flange 418 and secondary flange 420 have a chamfer on one edge creating an asymmetrical shape that is easy to insert into the main unit but provides greater resistance to removal to hold the main unit to the mouthpiece. Light blocking portion 404 may be formed integrally with light conduction portion 402 such as by overmolding. Light blocking portion 404 may be made of a white opaque flexible material such as rubber or silicone. The white material has the effect of reflecting and distributing light to the teeth while reducing or eliminating light shining through the cheeks of a user or escaping between the user's mouth and the main unit. A reflective material or coating may be placed between light conduction portion 402 and light blocking portion 404 for even greater effect.

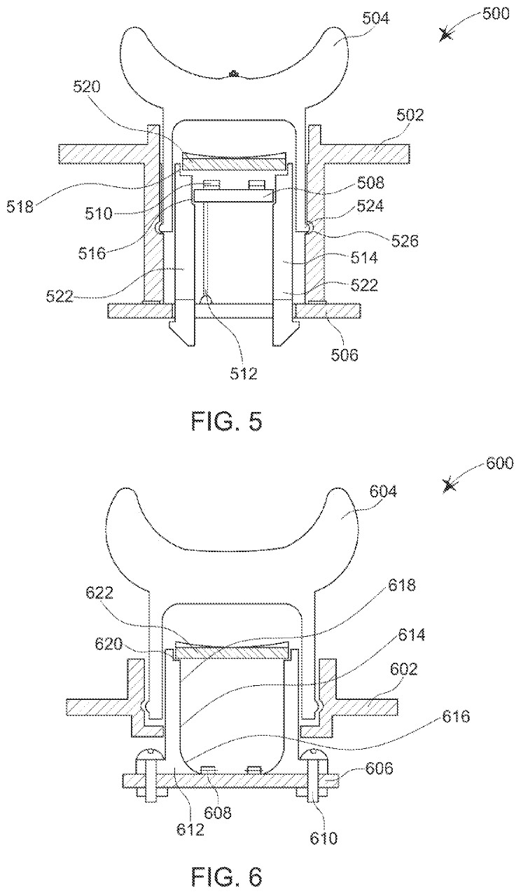

FIG. 5 depicts a sectional view of a portion of a teeth whitening apparatus 500 according to an embodiment. Teeth whitening apparatus 500 is comprised of a main unit 502 and a mouthpiece 504. Main unit 502 comprises a main printed circuit board (PCB) 506 and a smaller auxiliary PCB 508. The main PCB 506 comprises most of the electronic components while the auxiliary PCB 508 comprises Light Emitting Diodes (LEDs) 510. Auxiliary PCB 508 is electrically connected to the main PCB 506 via power wires 512 to power the LEDs 510. Auxiliary PCB 508 is held a distance away from main PCB 506 by standoff 514. Standoff 514 has a recess 516 sized to accept auxiliary PCB 508 and locate and align it and thus locate and align the LEDs. Auxiliary PCB 508 may be attached to the standoff 514 by press fit, adhesive, mechanical fastener, or any other means. Standoff 514 also has a recess 518 for receiving and locating transparent barrier 520. Transparent barrier 520 may be attached using adhesive sealant to retain transparent barrier 520 and create a seal between transparent barrier 520 and standoff 514. Transparent barrier 520 may have a light entrance area on one side and a light exit area on the opposite side and may be shaped so as to act as a lens to direct light in a desired pattern. Standoff 514 is attached to main PCB 506 via legs 522 which protrude through holes in main PCB 506. Legs 522 are in the form of snap hooks having a chamfered end and a retaining ledge that holds standoff 514 onto main PCB 506. Standoff 514 may also be attached using adhesive or may have sealant applied at the intersection of standoff 514 and main PCB 506 to seal the interior of the main unit. Mouthpiece 504 is removably attached to the main unit 502 via a toroidal protrusion 524 around the exterior end of the mouthpiece 504 which is designed to interface with a corresponding toroidal groove 526 in the main unit.

FIG. 6 depicts a sectional view of a portion of a teeth whitening apparatus 600 according to an embodiment. Teeth whitening apparatus 600 is comprised of a main unit 602 and a mouthpiece 604. Main unit 602 comprises a main printed circuit board (PCB) 606 having LEDs 608 mounted thereon. Reflector 612 is attached to main PCB 606 by fasteners 610 in the form of a screw and nut which pass through corresponding holes in the reflector 612 and main PCB 606. Reflector 612 may also be attached to main PCB 606 using adhesive. Reflector 612 has an opening sized to encompass all of the LEDs 608. Reflector 612 may have light colored, shiny, or reflective inside surface 614 to allow light to convey through the reflector and into the mouthpiece. In one embodiment the inside surface 614 is comprised of chromed plastic. The inside surface 614 is comprised of a curved lower portion 616 that meets up with straight upper portion 618. Curved lower portion 616 may collect light emanating at angles far from normal and redirect it in a more normal direction. Reflector 612 has a recess 620 for receiving and locating transparent barrier 622. Transparent barrier 622 may be attached using adhesive sealant to retain transparent barrier 622 and create a seal between transparent barrier 622 and reflector 612. Transparent barrier 622 may have a light entrance area on one side and a light exit area on the opposite side and may be shaped so as to act as a lens to direct light in a desired pattern.

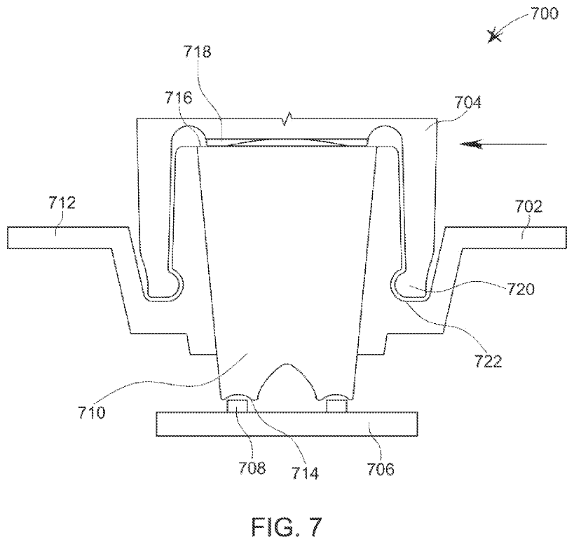

FIG. 7 depicts a sectional view of a portion of a teeth whitening apparatus 700 according to an embodiment. Teeth whitening apparatus 700 is comprised of a main unit 702 and a mouthpiece 704. Main unit 702 comprises a main printed circuit board (PCB) 706 having LEDs 708 mounted thereon. A light pipe 710 is attached to the housing 712 of the main unit 702. Light pipe 710 may be made of a transparent or translucent material such as glass or polycarbonate. A sealant or adhesive may be used to attach and seal light pipe 710 to housing 712. Light pipe 710 may use total internal reflection to guide light from LEDs 708 to the mouthpiece 704. Light pipe 710 may have one or more light entrance areas 714 in which light emitted by LEDs 708 is received into the light pipe. Opposite the light entrance area 714 is a light exit area 716 in which light exits the light pipe 710 and passes into the mouthpiece 704. Mouthpiece 704 may have a light entrance area 718 that is designed to receive light emitted from light exit area 716. Light entrance area 714, light exit area 716, and light entrance area 718 may be planar or may be shaped to direct light. For example, they may be concave or convex. Mouthpiece 704 is removably attached to the main unit 702 via a toroidal protrusion 720 around the interior end of the mouthpiece 704 which is designed to interface with a corresponding toroidal groove 722 in the housing of the main unit.

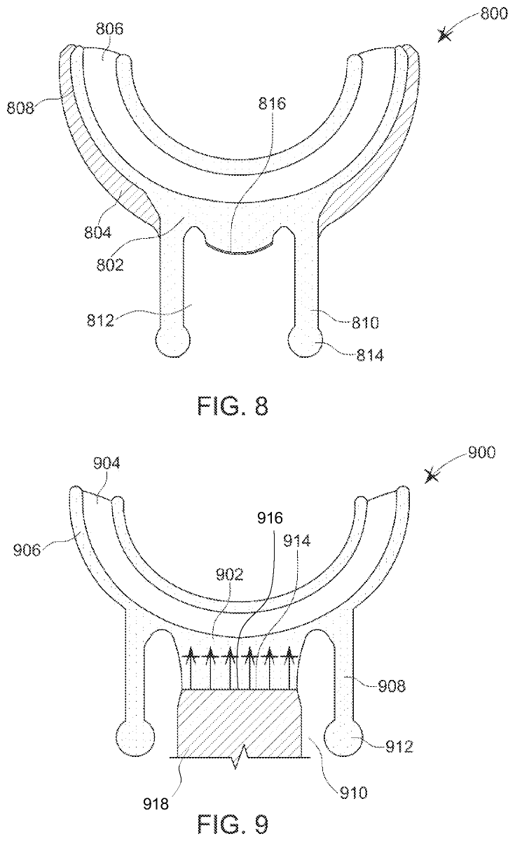

FIG. 8 depicts a mouthpiece 800 according to an embodiment. Mouthpiece 800 is comprised of a transparent or translucent (semi-transparent) light conducting portion 802 and an opaque or nearly opaque light blocking portion 804. Light conducting portion 802 is designed to transmit and disperse light from a light source of a main unit to the teeth of a user. Light blocking portion 804 is designed to prevent light from escaping the mouthpiece in undesired directions. Light conducting portion 802 is comprised of a bite section 806 having a planar crescent or partial ring shape that is designed to fit between upper and lower teeth of a user. An outside flange 808 extends perpendicular to and along the outer circumference the bite section 806 and is designed to extend along the front side of the user's teeth. Light blocking portion 804 encompasses the outer surface of outside flange 808. Light conducting portion 802 extends beyond light blocking portion 804 as tube 810 comprised of a hollow thin shape having an inner cavity 812 therein. Inner cavity 812 may receive a portion of a main unit therein. The end of tube 810 comprises a toroidal protrusion 814 which is designed to connect to a main unit. Light entrance area 816 has a convex shape which directs light in a desired direction.

FIG. 9 depicts a mouthpiece 900 according to an embodiment. Mouthpiece 900 is comprised of a transparent or translucent (semi-transparent) light conducting portion 902 which is designed to transmit and disperse light from the light source of the main unit to the teeth of a user. Light conducting portion 902 is comprised of a bite section 904 having a planar crescent or partial ring shape that is designed to fit between upper and lower teeth of a user. An outside flange 906 extends perpendicular to and along the outer circumference the bite section 904 and is designed to extend along the front side of the user's teeth. Light conducting portion 902 has tubular section 908 comprised of a hollow thin shape having an inner cavity 910 therein. Inner cavity 910 may receive a portion of a main unit therein. The end of tubular section 908 comprises a toroidal protrusion 912 which is designed to connect to a main unit. Light entrance area 914 has a planar shape and is designed to fit closely against a light exit area 916 of a main unit light pipe 918 to transmit light directly from the main unit to the light conducting portion 902 without passing through open air. Such as design may help contain light without the need for a light blocking portion or reflective ring.

A portable teeth whitening apparatus disclosed herein may include additional features. An air pump, blower, or the like may be used to generate airflow which is directed to the teeth in order to help keep the teeth cool or dry. The airflow can be directed from a pump through the mouthpiece and onto the teeth through dedicated channels or via the central opening in the mouthpiece. The air may have several beneficial effects including improving the effectiveness of the teeth whitening session, the user ingesting less whitening gel, and a more comfortable whitening session. Additionally, the air may allow the light output to be increased without causing damage to the teeth and gums due to heat, resulting in improved whitening performance. A temperature sensor may be included to measure the temperature of the teeth or gums to ensure that damage is not done. The temperature sensor may be an infrared sensor or any other suitable device.

The discussion herein of the present invention is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the description has broad application, and the discussion of any embodiment is meant only to be exemplary of that embodiment, and not intended to limit that the scope of the disclosure, including the claims, is limited to that embodiment.

Herein, the terms “including,” “consisting of”, and “comprising” are used in an open-ended fashion, and thus should be interpreted to mean “including, but not limited to.” Also, the term “connect” or “connected” where used if at all is intended to mean either an indirect or direct connection. Thus, if a first component connects to a second component, that connection may be through a direct connection or through an indirect connection via other components and connections.

Certain terms are used throughout the description and claims to refer to particular system components and method steps. As one skilled in the art will appreciate, different companies may refer to a component by different names. This document does not intend to distinguish between components that differ in name but not function.

It is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A portable teeth whitening apparatus comprising:
a main unit comprising:
a housing having an internal cavity therein;
a light source within the housing for emitting light to be applied to teeth of a user; and
a detachable mouthpiece that is removably connectable to the main unit, the mouthpiece designed to fit in the mouth of a user and allow at least a portion of light emitted from the light source to pass through the mouthpiece to the teeth of the user.

2. The portable teeth whitening apparatus of claim 1, wherein the mouthpiece is made of a translucent or transparent material.

3. The portable teeth whitening apparatus of claim 2, wherein the mouthpiece is made of silicone rubber.

4. The portable teeth whitening apparatus of claim 2, wherein the mouthpiece further comprises a reflective ring.

5. The portable teeth whitening apparatus of claim 1, wherein the mouthpiece comprises a light conducting portion and a light blocking portion, the light conducting portion comprising a first material that is translucent and the light blocking portion comprising a second material that is opaque.

6. The portable teeth whitening apparatus of claim 5, wherein the mouthpiece comprises a tubular section for interfacing with the main unit and a U-shaped section to be placed in the user's mouth.

7. The portable teeth whitening apparatus of claim 6, wherein the U-shaped section comprises an outside flange and wherein the light blocking portion extends along an outer surface of the tubular section and along an outer surface of the outside flange.

8. The portable teeth whitening apparatus of claim 5, wherein the mouthpiece further comprises a locking flange designed to secure the mouthpiece to the main unit.

9. The portable teeth whitening apparatus of claim 5, wherein the first material and second material are silicone rubber.

10. The portable teeth whitening apparatus of claim 5, wherein the second material is white.

11. A portable teeth whitening apparatus comprising:
a housing having an internal cavity therein;
a light source within the housing for emitting light to be applied to teeth of a user; and
a mouthpiece connected to the housing, the mouthpiece designed to fit in the mouth of a user and allow at least a portion of light emitted from the light source to pass through the mouthpiece to the teeth of the user,
wherein the light source is designed to emit light according to a lighting scheme, the lighting scheme defining the intensity of the light source during a treatment session, and
wherein the lighting scheme has a variable intensity during the treatment session.

12. The portable teeth whitening apparatus of claim 11, wherein the lighting scheme comprises a repeating waveform.

13. The portable teeth whitening apparatus of claim 12, wherein the waveform has a wavelength of one to three seconds.

14. The portable teeth whitening apparatus of claim 12, wherein the waveform is a square wave.

15. The portable teeth whitening apparatus of claim 14, wherein the waveform has a wavelength of one to three seconds.

16. The portable teeth whitening apparatus of claim 14, wherein the waveform has a wavelength of two seconds, the light being at a first intensity for one second and at zero intensity for one second.

17. The portable teeth whitening apparatus of claim 12, wherein the waveform comprises a sine, square, sawtooth, or triangle wave.

18. The portable teeth whitening apparatus of claim 17, wherein the waveform has a wavelength of one to three seconds.

19. The portable teeth whitening apparatus of claim 11, wherein the apparatus comprises a plurality of lighting schemes and the user may select a lighting scheme from the plurality of lighting schemes.

20. The portable teeth whitening apparatus of claim 11, further comprising:
a safety sensor to reduce or halt light output of the light source when the portable teeth whitening apparatus is not in proximity to the user.
